SEQUENCE LISTING (7 sequences)

SEQ ID NO: 1 - protein, 221 aa, Zingiber officinale
DDLPDSIDWRENGAVVPVKNQGGCGSCWAFSTVAAVEGINQIVTGDLISLSEQQLVDCTTANHGCRGGWMNPAFQFIVNNGGINSEETYPYRGQDGICNSTVNAPVVSIDSYENVPSHNEQSLQKAVANQPVSVTMDAAGRDFQLYRSGIFTGSCNISANHALTVVGYGTENDKDFWIVKNSWGKNWGESGYIRAERNIENPDGKCGITRFASYPVKKGTN

SEQ ID NO: 2 - protein, 221 aa, Zingiber officinale
  MISC_FEATURE (219)..(219): X = any amino acid
DVLPDSIDWREKGAVVPVKNQGGCGSCWAFDAIAAVEGINQIVTGDLISLSEQQLVDCSTRNHGCEGGWPYRAFQYIINNGGINSEEHYPYTGTNGTCDTKENAHVVSIDSYRNVPSNDEKSLQKAVANQPVSVTMDAAGRDFQLYRNGIFTGSCNISANHYRTVGGRETENDKDYWTVKNSWGKNWGESGYIRVERNIAESSGKCGIAISPSYPIKEXXX

SEQ ID NO: 3 - protein, 469 aa, H5N1 influenza virus
MNPNQKITTIGSICMVIGIVSLMLQIGNIISIWVSHSIQTGNQHQAEPCNQSIITYENNTWVNQTYVNISNTNFLTEKAVNLVTLAGNSSLCPISGWAVYSKDNGIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYDGIITDTIKSWRNNILRTQESECACVNGSCFTVMTDGPSNGQASYKIFRIEKGKVVKSAELNAPNYHYEECSCYPDAGEITCVCRDNWHGSNRPWVSFNQNLEYRIGYICSGVFGDNPRPNDGTGSCGPVSPKGAYGIKGFSFRYGNGVWIGRTKSTNSRSGFEMIWDPNGWTGTDSNFSVKQDIVAITDWSGYSGSFVQHPELTGLDCIRPCFWVELIRGRPKESTIWTSGSSISFCGVNSDTVGWSWPDGAELPFTIDK

SEQ ID NO: 4 - protein, 567 aa, H5N1 influenza virus
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVMWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 5 - protein, 568 aa, H5N1 influenza virus
MEKIVLLLAIVSLVKSDQICIGYHANNSTEQVDTIMEKNVTVTHTQDILEKKHNGKLCDLDGVKPLILRDCSVAGWLLGNPMCDEFINVPEWSYIVEKASPANGLCYPGDFNDYEELKHLLSRINHFEKIQIIPKSSWSNHEASSGVSSACPYQGRSSFFRNVVWLIKKNGAYPTIKRSYNNTNQEDLLVLWGIHHPNDAAEQTKLYQNPTTYISVGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLNREEISGVKLESIGTYQILSIYSTVASSLALAIMVAGLSLWMCSNGSLQCRICI

SEQ ID NO: 6 - protein, 501 aa, H5N1 influenza virus
DQICIGYHANNSTEQVDTIMEKNVTVTHAQDILEKTHNGKLCDLNGVKPLILRDCSVAGWLLGNPMCDEFLNVPEWSYIVEKDNPVNGLCYPEDFNDYEELKHLLSSTNHFEKIRIIPRSSWSNHDASSGVSSACPYNGRSSFFRNVVWLIKKNNAYPTIKRSYNNTNQEDLLILWGIHHPNDAAEQTKLYQNPTTYVSVGTSTLNQRSVPEIATRPKVNGQSGRMEFFWTILKPNDAINFESNGNFIAPEYAYKIVKKGGSAIMKSGLEYGNCNTKCQTPMGAINSSMPFHNIHPLTIGECPKYVKSGRLVLATGLRNVPQRETGLFGAIAGFIEGGWQGMVDGWYGYHHSNEQGSGYAADKESTQKAIDGTTNKVNSIIDKMNTQFEAVGKGFNNLERRIENLNKKMEDGFLDVWTYNAELLVLMENERTLDFHDSNVKNLYDKVRLQLRDNAKELGNGCFEFYHKCDNECMESVKNGTYDYPQYSEEARLNREEISGV

SEQ ID NO: 7 - protein, 503 aa, H3N2 influenza virus
QDLPGNDNSTATLCLGHHAVPNGTLVKTITDDQIEVTNATELVQSSSGKICNNPHRILDGIDCTLIDALLGDPHCDVFQNETWDLFVERSKAFSNCYPYDVPDYASLRSLVASSGTLEFITEGFTWTGVIQNGGSNACKRGPGSGFFSRLNWLTKSGSTYPVLNVTMPNNDNFDKLYIWGIHHPSTNQEQTSLYVQASGRVTVSTRRSQQTIIPNIGSRPWVRGLSSSRISIYWTIVKPGDVLVINSNGNLIAPRGTFKMRTGKSSIMRSDAPIDTCISECITPNGSIPNDKPFQNVNKITYGACPKTVKQNTLKLATGMRNVPEKQTGLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEEMGNGCFKIYHKCDNACIESIRNGTYDHDVYRDEALNNRFQIKG

(19) United States
(12) Patent Application Publication
(10) Pub. No.: US 2009/0186012 A1
(43) Pub. Date: Jul. 23, 2009

(54) PLANT EXTRACT

(75) Inventor: Clifford J. Hawkins, Queensland (AU)

Correspondence Address: KNOBBE MARTENS OLSON & BEAR LLP, 2040 MAIN STREET, FOURTEENTH FLOOR, IRVINE, CA, 92614, US

(73) Assignee: Natbio Pty. Ltd., Kangaroo Point, Queensland (AU)

(21) Appl. No.: 12/093,938

(22) PCT Filed: Nov. 16, 2006
(86) PCT No.: PCT/AU2006/001717

§ 371 Date: Sep. 9, 2008

(30) Foreign Application Priority Data
Nov. 16, 2005 (AU) 2005906363

(51) Int. Cl. A61K 38/48; A61P 31/16

(52) U.S. Cl. 424/94.63

(57) ABSTRACT

The present invention relates generally to plant extracts and/or components isolated therefrom which exhibit desirable properties in relation to therapy. More particularly, the present invention relates to extracts and components isolated thereof from the plant genus Zingiber and in particular from the rhizome of the species Zingiber officinale (also known as ginger) which comprise activities having broad applicability in the field of inhibition and treatment of infections by pathogenic micro-organisms including viruses, bacteria, protozoa and parasites.

### BACKGROUND OF THE INVENTION

1. Field of the Invention

The present invention relates generally to plant extracts and/or components isolated therefrom which exhibit desirable properties in relation to therapy. More particularly, the present invention relates to extracts and components isolated thereof from the plant genus Zingiber and in particular from the rhizome of the species Zingiber officinale (also known as ginger) which comprise activities having broad applicability in the field of inhibition and treatment of infections by pathogenic micro-organisms including viruses, bacteria, protozoa and parasites.

2. Description of the Prior Art

Bibliographic details of the publications referred to by author in this specification are collected at the end of the description.

Reference to any prior art in this specification is not, and should not be taken as, an acknowledgment or any form of suggestion that this prior art forms part of the common general knowledge in any country.

Extracts from the tissues of monocotyledonous and dicotyledonous plants have provided a vast number of compounds and mixtures of compounds useful in medicine—including both Western-style and traditional approaches, such as those used in Sharmanism and Chinese medicine.

The rhizome of the ginger plant, Zingiber officinale, has been used as a spice in food preparation and as a non-specific “herbal remedy” for various disease conditions, sometimes in conjunction with honey. Neither the efficacy nor the underlying activity, however, has been delineated or quantified in a manner permitting reliable reproducible outcomes, sufficient for consistent treatment purposes. Furthermore, studies designed to assess such presumptions have had to contend with the over-riding difficulty of lack of consistent and reproducible trial data.

Surprisingly, the subject inventors have identified a number of useful and varied applications for the various ginger rhizome extracts and/or components thereof. In accordance with the present invention, the difficulties associated with variability and lack of consistency of various extracts and components of the ginger rhizome have been overcome. This has enabled the quantification and characterization of the ginger rhizome and extracts thereof and its components.

Avian influenza viruses infect hosts in a highly species specific manner, but on rare occasion have crossed the species barrier to infect humans. A highly pathogenic avian influenza has caused outbreaks in various countries and is considered endemic in Indonesia, Vietnam, and some parts of Cambodia, China and Thailand. The H5N1 avian influenza A virus is highly pathogenic and has been shown to lead to an extreme elevation of inflammatory cytokines with rapid deterioration and high fatality. Many of those infected by H5N1 suffer from severed respiratory diseases such as pneumonia and multi-organ failure. Neuraminidase inhibitors which reduce the severity and duration of human influenza, could be used to treat avian influenza virus. However, they have substantial constraints, namely that production capacity of these drugs is limited and too expensive for many countries.

Because of the outbreaks of avian influenza and a lack of anti-viral agent there is a need for agents which can inhibit viruses and in particular the avian influenza virus.

In accordance with the present invention, ginger rhizome extracts and/or components thereof has surprisingly been found to inhibit both human and avian influenza virus subtypes, even though the protein structure of the human influenza virus does not have a high level of homology with important protein components of the avian influenza virus.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1 (SEQ ID NO: 1), <400>2 (SEQ ID NO:2), etc. A summary of the sequence identifiers is provided in Table 1. A sequence listing is provided at the end of the specification.

The present invention provides extracts, and components thereof, derived from members of the Zingiberaceae plant family. Members of this family include, for example, Zingiber mioga, Zingiber officinale, Zingiber cassumunar and Zingiber zerumbet. Reference to “Zingiber extracts” in the subject specification includes extracts from all of the aforementioned species. The preferred species is Zingiber officinale, also known as ginger. The extracts and components, derived from the rhizome of the Z. officinale plant, comprise activities which are able to be applied usefully in disease prophylaxis and treatment. One particular example of a disease state is infection by a virus, bacteria, protozoa, parasites, or eukaryotic organism (e.g. fungus, yeast, lower eukaryote).

The useful activities are found in one or more fractions derived from finely cutting and extracting (“crushing”) the ginger (Zingiber) rhizome. The resulting crush may be dried to generate an active powder form or, alternatively, may be filtered to produce a crush filtrate from which may be generated an “isolate” comprising the components referred to herein as “Zingibain”. In any of the foregoing cases—the crush, the dried powder, the crush filtrate or the isolate—the preferred active component comprised therein is Zingibain. Zingibain activity may be used consistently and reliably to hydrolyze, in a highly specific manner, a particular target. More particularly, Zingibain is effective in any situation wherein the target comprises a proteinaceous molecule that comprises a significant percentage of proline residues. The proline residues are preferably preceded and/or followed by a hydrophilic amino acid. Suitable amino acids include, for example, glutamine, arginine, lysine, asparagine, glutamic acid and aspartic acid. Reference herein to “Zingibain activity” may also be read as “Zingibain activities”.

As stated above, the preferred active component comprised within the extract or a molecular component thereof is referred to herein as “Zingibain” and it has application in a wide range of related fields.

One field of application wherein the Zingibain activity of the present invention finds use is in the maintenance of health of animals, including human animals as well as performance, companion and farm animals, and to prophylaxis and treatment of diseases/disorders of animals including humans.

The plant extract of the instant invention is further useful as a medicament or in the manufacture of a medicament for the treatment and, in some cases, prophylaxis of infection by viruses, bacteria, protozoa, parasites, fungi, yeast or lower eukaryotes.

Skin disorders of the foregoing type typically involve superficial lesions and/or abnormalities that require topical application of a medicament useful in the treatment thereof. The instant invention, however, provides agents that may be formulated as medicaments for systemic administration, for example, as a powder, liquid, syrup, tablet, and capsule. Hence, the extract and/or molecular components thereof are applicable for treatment and, in some cases, prophylaxis of infection by pathogenic organisms including viruses.

In related embodiments, the extract and/or molecular components of the present invention may be applied in a method for the prevention and/or treatment of a range of disease states, including a systemic and/or skin disorder such as those recited above.

The extract and components of the present invention exhibit proteolytic activity directed, in particular, at targets adjacent to a conformationally exposed proline residue preceded and/or followed by a hydrophilic amino acid residue. Thus, Zingibain may be used consistently and reliably to hydrolyze such a target.

Hence, one aspect of the present invention is directed to the use of a rhizome from a species of Zingiber in the manufacture of an extract or a molecular component or fraction thereof which hydrolyzes proline-contain proteins.

Another aspect of the present invention provides for the use of a rhizome from Zingiber officinale in the manufacture of a medicament for the treatment of infection by influenza virus.

Yet another aspect of the present invention contemplates a method of treatment of a disease comprising administering to a person a therapeutically effective amount of an extract of Zingiber comprising at least one cysteine protease.

Still yet another aspect of the present invention is directed to a use of an extract of Zingiber comprising at least one cysteine protease or a composition comprising the same for the manufacture of a medicament for the treatment of an infection caused by a pathogenic agent.

Even another aspect of the present invention provides a pharmaceutical formulation and/or health supplement formulation comprising an extract of Zingiber comprising at least one cysteine protease.

TABLE 1
Summary of sequence identifiers

| Sequence | |
|---|---|
| ID NO: | Description |
| 1 | Amino acid sequence of the component, isolatable |
| | from the ginger rhizome fraction designated GP-II, |
| | and exhibiting cysteine protease activity |
| 2 | Amino acid sequence of the dominant component, isolatable |
| | from the ginger rhizome fraction designated GP-I, and |
| | exhibiting cysteine protease activity |
| 3 | Amino acid sequence of neuraminidase N1 found in |
| | H5N1 avian influenza virus |
| 4 | Amino acid sequence of H5 Hemagglutinin found in |
| | Vietnam H5N1 avian influenza virus |
| 5 | Amino acid sequence of H5 Hemagglutinin found in |
| | Japan/China H5N1 avian influenza virus |
| 6 | Amino acid sequence of H5 Hemagglutinin found in |
| | Singapore H5N1 avian influenza virus |
| 7 | Amino acid sequence of H3 Hemagglutinin found in |
| | H3N2 influenza virus |

### BRIEF DESCRIPTION OF THE FIGURES

FIG. 1 is a representation of the structure of Zingibain, showing the four molecules in the crystallobraphic unit cell in two different orientations with the helical domains represented by cylindrical tube like shapes and the β-sheet domains represented by flat rectangular shapes. The locations of the saccharide moieties are also indicated (Choi et al, Biochem. 38:11624-11633, 1999).

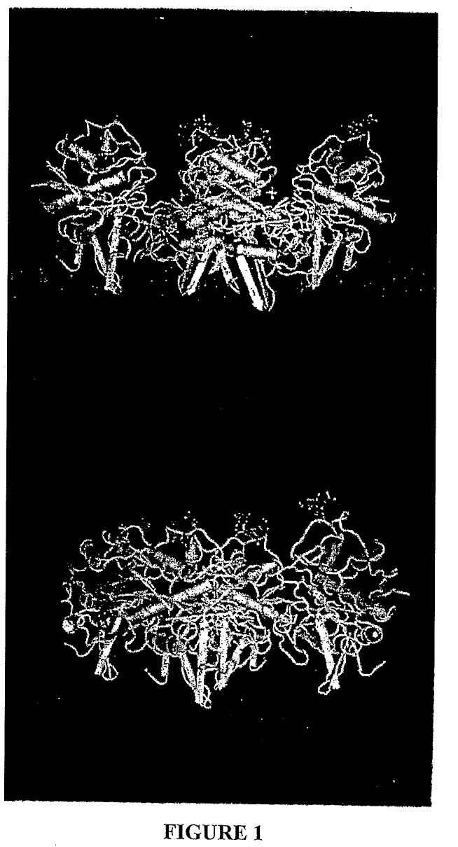

FIG. 2 is a representation of the structure of H5 Hemagglutinin for the Japan/China variant.

FIG. 3 is a graphical representation showing the loss of H5N1 infectivity after preincubation with Zingibain extract at 37° C.

### DETAILED DESCRIPTION OF THE INVENTION

The present invention is predicated in part on the observation that members of the Zingiberaceae plant family comprise an extractable fraction, which fraction or components thereof exhibit properties useful in a range of applications. The preferred species is Zingiber officinale, also known as ginger. Other members of the Zingiberaceae plant family are, however, not precluded and are intended to fall within the scope of the present invention. Examples of other species of Zingiber include Z. mioga, Z. cassumunar and Z. zerumbet. Reference hereinafter to a “ginger plant” is not intended to exclude species of Zingiber other than Z. officinale.

Preferably, the species of Zingiber is Z. officinale.

The present invention identifies and delineates a wide range of useful applications for definable extracts and components from Zingiber such as Z. officinale. The extracts and components thereof are found to comprise inter alia hydrolytic activity, capable of acting highly specifically on proteinaceous molecules that comprise a conformationally exposed proline residue preceded and/or followed by a hydrophilic amino acid residue.

Reference to “Z. officinale” or “Zingiber officinale ” or “ginger plant” is to be read as including other species or genera of the Zingiberaceae family which have similar properties.

The “molecular components”, which are comprised in and isolatable from an extract of the Zingiber, such as Z. officinale, rhizome, are enzymes the function of which is to degrade proteins. These enzymes are generally referred to as proteolytic enzymes or proteases, and they function by hydrolyzing peptide bonds within the amino acid, sequences that constitute proteins. As will be well known to one skilled in the art, proteases are ubiquitous in nature and are many and varied in their structure and particular preferred substrate. They are, therefore, generally grouped into like kinds, according to their usual target.

One group of proteases is referred to in the art as “cysteine proteases”, in which a thiol group of a cysteine residue is the nucleophilic group involved in attacking and hydrolyzing a peptide bond. Representative members of the “cysteine protease” group of proteolytic enzymes include, for example, papain, bromelain and ananain, ficin and actinidin. These molecules are isolatable from papaya (Carica papaya), pineapple (Ananas comosus), figs, and kiwi-fruit (Actinidin chinensis), respectively. Zingibain is from the group of enzymes known as “cysteine protease”. More particularly, Zingibain is a proline-specific cysteine protease. Accordingly, Zingibain is effective in any situation wherein the target is a proteinaceous molecule that comprises a significant percentage of proline residues.

In the context of the present invention, “significant percentage” is to be understood as an amount of proline in excess of about 5%, which is higher than normal in proteins and which gives a greater chance of proline being preceded or followed by a hydrophilic amino acid residue in an exposed site for successful hydrolysis.

Preferably, the percentage of proline is less than about 60%, more preferably less than about 50%, even more preferably less than about 40%, still more preferably less than about 30%, and most preferably less than about 20%. Hence, a reference herein to a “proline-containing protein” is to be understood to be a reference to a proteinaceous molecule that comprises a significant percentage of proline residues, as hereinbefore defined.

Accordingly, one aspect of the present invention is directed to the use of a rhizome from a species of Zingiber, preferably Z. officinale rhizome, in the manufacture of an extract or a molecular component thereof, which is capable of hydrolyzing proline-containing proteins including protein fragments (peptides).

The molecular components that provide the useful activity of the present invention are found in a fraction derived from finely cutting or otherwise comminuting rhizome of Zingiber such as Z. officinale.

A number of different formulations may be derived from processing the finely cut ginger rhizome. The cut tissue may be dried to generate the spicy ginger known to culinary aficionados. Alternatively, the finely cut rhizome may be extracted to produce a “ginger crush”, the solution of which comprises the desired active molecular components of the present invention.

This ginger crush may be dried to generate an active powder form or, alternatively, may be filtered to produce a crush filtrate from which may be isolated Zingibain, which is regarded herein as one of the molecular components of the ginger plant extract. In any of the foregoing formulations—the dried powder, the crush or its filtrate or the isolate—the preferred activity is due to a Zingibain extract. Reference herein to “molecular components” includes a component or extract having the characteristics of Zingibain.

“Zingibain”, as used herein, refers to a protein fraction, isolatable from ginger rhizome, and comprising proteolytic activities of at least one or two or three closely related enzyme fractions, separable by, for example, DEAE-cellulose chromatography. One of the fractions comprises the GP-II proteases. Another fraction, referred to as “GP-I”, comprises two highly homologous proteases. All three proteolytic enzymes are comprised in the dried powder, the crush or its filtrate or the isolate as described herein. Hence, reference to “molecular components” is a reference to any one of or, alternatively, all three proteolytic enzymes. Similarly, throughout this specification, a reference to “Zingibain” is to be understood to be a reference to the unseparated protease fraction comprising all three protease enzyme activities, or to any one or more of the said protease activities.

Without intending to limit the present invention to any one theory or mode of action, it is proposed that Zingibain degrades its protein targets by hydrolyzing peptide bonds between an amino acid residue following a proline and the next amino acid residue thereafter in the amino acid sequence, reading from the N-terminal.

For optimal hydrolytic effect, a proline residue is preferably preceded and/or followed by a hydrophilic amino acid. Suitable hydrophilic amino acid residues include, for example, glutamine, arginine, lysine, asparagine, glutamic acid and aspartic acid.

The term “hydrolyzing” as applied to the effect of a proteolytic enzyme on a peptide bond means that the affected peptide bone is broken or destroyed, and the sequence of the hydrolyzed protein is thereby severed or cleaved at that point in the chain. An attacked protein may be broken down, through hydrolysis, into two or into many peptide pieces, depending on the extent of suitable bonds for hydrolysis and on the extent of hydrolysis that actually occurs. Hydrolysis, therefore, destroys proteinaceous material and results in its conversion and/or degradation into smaller cleaved portions of protein, or peptides, and/or, in its most extreme form, into the amino acid constituents thereof. Destroyed, degraded, converted, cleaved, and/or hydrolyzed proteinaceous material no longer exhibits its naturally occurring function.

Zingibain's specificity for hydrolyzing proteins adjacent to proline results in the splitting of the protein rather than in the break-down of proteins to small peptide pieces or individual amino acids. However, if the substrate for the Zingibain is a peptide, for example, which is the product of digestion/hydrolysis of other proteolytic enzymes such as those found in the stomach including enzymes like trypsin and/or chymotrypsin, then the product after hydrolysis with Zingibain may be individual amino acids and/or di- or tri-peptides.

In accordance with the present invention, ginger rhizomes may be processed to generate extracts that comprise the proline-specific cysteine protease, Zingibain, which is capable of destroying and/or degrading proteins via hydrolysis adjacent to or “following” proline residues.

As used herein, the term “extract” extends to and encompasses any formulation, derived from the Zingiber, such as Z. officinale, rhizome, in which Zingibain exists and may be used in accordance with the present invention. “Extract” therefore extends to dried, powder, ginger crush, crush filtrate and isolate, as described above, and any other suitable formulation. The terms “extract” and “Zingibain” are used herein interchangeable.

The uses according to the present invention include use of a protein substantially identical to Zingibain regardless of its source, for example, regardless of whether it is prepared recombinantly, synthetically and/or probiotically in situ. Substantially identical in the context of this specification will be understood to mean at least 95% identity with the sequences No. 1 or No. 2 at the amino acid level. Preferably substantially identical will be 98% identity and more preferably 99% identity.

Thus the subject invention extends to compositions, preferably pharmaceutical and/or health supplement formulations comprising or consisting of said enzyme/extract. The enzyme extract may be formulated as a tablet, capsule, powder, drink or the like. However, the enzyme may need protecting to survive to the acidic conditions of the stomach, for example, by enteric coating or buffering.

Alternatively the enzyme may be made in situ in the gut by yeast or bacteria designated/engineered to synthesise the enzyme/components.

The yeast and/or bacteria may be administered in the form of an active drink. Thus the invention also extends to probiotic formulations capable of preparing the enzyme in vivo. Probiotic formulations according to the invention may be a fermented product derived from milk or soy or similar. Such formulations may include lactose, glucose, stabilisers and one or more flavourings. Yeast and bacteria which may be employed in the probiotic formulations are known to persons skilled in the art. Preferably the bacteria are Lactobacillus such as Lactobacillus casei.

Compositions as referred to herein are characterised by the presence of one or more excipients such as a diluent or carrier.

Without limiting the invention to any one theory or mode of action, it is proposed that the extract or molecular component of Zingiber, such as Z. officinale, rhizome specifically hydrolyzes proteins that comprise a significant percentage of proline residues. Particularly preferred proline-rich natural proteins include, but are not limited to, particular cell membrane proteins including receptors etc., inter alia. Since these molecules are involved in many cellular and biochemical processes, the ginger rhizome extract referred to herein as Zingibain has applications, even more widely, in preventing and/or treating the effects of biochemical processes that may be undesirable and/or deleterious to health. Such processes may be superficial—affecting, for example, skin—or they may be systemic.

The plant extract of the instant invention is therefore useful as a medicament or in the manufacture of a medicament for the treatment and, in some cases, prophylaxis of a disease condition or disease condition of the skin caused by microbial infection where the infective agent has cell membrane proteins with proline adjacent to a hydrophilic amino acid, which are implicated in the infectivity or in the damaging immune response to the invading organism.

Accordingly, another aspect of the present invention is directed to the use of an extract of the Z. officinale rhizome, wherein said extract comprises molecular components capable of hydrolyzing proline-containing proteins, in the preparation of a medicament for the prophylaxis and/or treatment of a skin disorder or other disorder described herein, in a subject. Such disorders include, for example, microbial infection where the infective agent has cell membrane proline containing proteins that have an exposed site for hydrolysis. Examples of such disorders include, for example, diseases caused by pathogenic organisms including viruses such as the influenza virus, protozoa, parasites and bacteria.

Reference herein to “prophylaxis” and “treatment” is to be considered in its broadest context. The term “treatment” does not necessarily imply that a subject is treated until total recovery. Similarly, “prophylaxis” does not necessarily mean that the subject will not eventually contract a disease condition. Accordingly, prophylaxis and treatment includes amelioration of the symptoms of a particular disorder or condition, or preventing or otherwise reducing the risk of developing a particular disorder or condition. The term “prophylaxis” may be considered as reducing the severity or the onset of a particular disorder. “Treatment” may also reduce the severity of an existing condition.

In this context, a “subject” may be a human or an animal subject.

Skin disorders of the foregoing type, which may be amenable to prophylaxis and/or treatment in this manner, typically involve superficial lesions and/or abnormalities that require topical application of a medicament useful in the treatment thereof. Such disorders include, for example, infections by pathogenic micro-organisms.

Accordingly, yet another aspect of the present invention is directed to a method of treating and/or preventing a skin disease and/or abnormality in a subject, said method comprising contacting said diseased and/or abnormal skin with an effective amount of a medicament comprising an extract of the Z. officinale rhizome, wherein said extract comprises molecular components capable of hydrolyzing proline-containing proteins, for a time and under conditions sufficient to prevent, ameliorate or otherwise reduce symptoms of said disease and/or abnormality.

n this regard, the instant invention provides agents that may be formulated as medicaments for systemic administration. Hence, the extract and/or molecular components thereof are also applicable for treatment and, in some cases, prophylaxis of a broader range of ailments including pathogenic micro-organisms such as viruses, bacteria, protozoa and parasites. Viruses, for example, include rhinoviruses, respiratory syncytial viruses, corona viruses, arboviruses, rotaviruses, para-influenza viruses and influenza viruses. Corona viruses include for example severe acute respiratory syndrome (SARS). Influenza viruses include for example, all mammalian influenza viruses which include but are not limited to equine influenza viruses, swine influenza viruses, human influenza viruses and avian influenza viruses or hybrids thereof.

Accordingly, a further aspect of the present invention contemplates the use of Z. officinale rhizome in the manufacture of a medicament comprising an extract or a molecular component thereof, which is capable of hydrolyzing proline-containing proteins, for the prophylaxis and/or treatment of a systemic disorder in a subject.

Thus in one embodiment, the present invention contemplates a method of treating and/or preventing a systemic disorder, said method comprising administering to a subject in need thereof an effective amount of a medicament comprising an extract of the Z. officinale rhizome, wherein said extract comprises molecular components capable of hydrolyzing proline-containing proteins, for a time and under conditions sufficient to prevent, ameliorate or otherwise reduce symptoms of the disorder.

Virus cell-membrane proteins commonly are proline-rich and have multiple sites for hydrolysis by Zingibain. These proteins are essential for host-cell invasion and other functions, and their cleavage by Zingibain inhibits the viral infection. Examples of such proteins are hemagglutinin and neuraminidase proteins of the influenza virus. Influenza viruses include all mammalian influenza viruses which include but are not limited to equine influenza viruses, swine influenza viruses, human influenza viruses and avian influenza viruses. Preferably, the avian influenza virus is the H5N1 avian influenza virus. Even more preferable are hemagglutinins including the H5 hemagglutinin found in the Singapore, Vietnam and Japan/China H5N1 avian influenza virus.

Without wishing to limit the present invention to any one theory or mode of action, it is proposed that hydrolysis of a viral cell-membrane protein with a proline adjacent to a hydrophilic amino acid by Zingibain inhibits viral infection and proliferation, which removes the antigenicity of the hemagglutinin protein and the associated T-cell release of damaging high levels of cytokines.

The active component of the medicament is contemplated to exhibit therapeutic activity when administered in an “effective amount” that depends on the particular case. By “effective amount” is meant an amount necessary to at least partly obtain the desired response, or to delay the onset or inhibit progression or halt altogether the onset or progression of a particular condition being treated. The amount varies depending upon the health and physical condition of the subject being treated, the taxonomic group of the subject being treated, the degree of protection desired, the formulation of the composition, the assessment of the medical situation and other relevant factors. It is expected that the amount will fall in a relatively broad range, which may be determined through routine trials. Considering a human subject, for example, from about 1 unit to about 10,000 units of protease activity may be administered per mL of solution or 1 unit to about 500,000 units of protease activity per gram dry powder or per gram honey or gram ointment per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

In accordance with the applications of the present invention, medicaments comprising the extracts and or components disclosed herein may be formulated, for use in conjunction with the instant methods, via topical administration or via systemic administration, depending on the nature of the subject's disorder. Appropriately formulated medicaments may then be utilised in the treating and/or preventing disease, whether a skin abnormality or disease, or a systemic disorder such as those referred to above. Such medicaments may be administered to a subject in any one of a number of conventional dosage forms and by any one of a number of convenient means. As already mentioned, “subject” may refer to any animal including but not limited to a human.

Contemplated suitable dosage forms of the active component include solutions, tablets, troches, pills, capsules, creams, oils, gels and the like, all of which may also contain additional components, as follows: a binder such as gum, acacia, corn starch or gelatin; excipients such as dicalcium phosphate; a disintegrating agent such as corn starch, potato starch, alginic acid and the like; a lubricant such as magnesium stearate; a sweetening agent such as sucrose, lactose or saccharin; a flavouring agent such as peppermint, oil of wintergreen or cherry flavouring. When the dosage unit form is a capsule, it may contain, in addition to materials of the above type, a liquid carrier. Various other materials may be present as coatings or to otherwise modify the physical form of the dosage unit. For instance, tablets, pills or capsules may be coated with shellac, sugar or both. A syrup or elixir may contain the active compound, sucrose as a sweetening agent, methyl and propylparabens as preservatives, a dye and flavouring such as cherry or orange flavour. Honey or molasses may contain an active compound. Of course, any material used in preparing any dosage unit form should be pharmaceutically pure and substantially non-toxic in the amounts employed. In addition, the active compound(s) may be incorporated into sustained-release preparations and formulations

Pharmaceutically acceptable carriers and/or diluents include any and all solvents, dispersion media, coatings, anti-bacterial and anti-fungal agents, isotonic and absorption delaying agents and the like. The use of such media and agents for pharmaceutical active substances is well known in the art and except insofar as any conventional media or agent is incompatible with the active ingredient, its use in the therapeutic composition is contemplated. Supplementary active ingredients can also be incorporated into the compositions.

The active component may be administered in a convenient manner such as by oral, intravenous (where water-soluble), intra-peritoneal, intramuscular, subcutaneous, intra-dermal or suppository routes, or via implanting (e.g. using slow release molecules).

Suitable amounts of active ingredient for oral dosage forms may include 1 Unit to 10,000 Units of protease activity per 10 Kg body weight, such as 10 to 1,000 Units of protease activity per 10 Kg body weight, and such as 100, or 500 Units. A preferred daily dosage is 3000-6000 Units per 10 Kg body weight.

Alternatively, the active component may be formulated for administration topically, such as by cream, oil or gel. The active component may be administered in the form of pharmaceutically acceptable non-toxic salts, such as alkali or alkaline earth salts, such as sodium, potassium, magnesium or calcium. The active component may be administered as a supplement to prepared food or drink.

Preferred formulations for topical administration include those in which the active component of the present invention is in admixture with a topical delivery agent such as lipids, liposomes, fatty acids, fatty acid esters, steroids, chelating agents and surfactants.

Preferred lipids and liposomes include neutral (e.g. dioleoylphosphatidyl DOPE ethanolamine, dimyristoylphosphatidyl choline DMPC, distearoylphosphatidyl choline), negative (dimyristoylphosphatidyl glycerol DMPG) and cationic (dioleoyltetramethyl-aminopropyl DOTAP and dioleoylphosphatidyl ethanolamine DOTMA).

For topical or other administration, the extract and/or components of the present invention may be encapsulated within liposomes or may form complexes thereto, in particular to cationic liposomes. Alternatively, the extract and/or component may be complexed to lipids, in particular to cationic lipids. Preferred fatty acids and esters, pharmaceutically acceptable salts thereof, and their uses are known, such as are described in U.S. Pat. No. 6,287,860.

In a related embodiment, the present invention contemplates the use of Z. officinale rhizome in the manufacture of an extract or a molecular component thereof, which is capable of hydrolyzing proline-containing proteins, for specific cleavage of an identified target.

The invention also contemplates a vaccine for stimulating a host's immune system, comprising a composition comprising a surface component of a virus, wherein said surface component has been treated with an extract of Z. officinale rhizome and said vaccine optionally further comprising one or more pharmaceutically acceptable carriers, adjuvants and/or diluents. Preferably, the virus is selected from the group consisting of equine influenza virus, swine influenza virus, human influenza virus and avian influenza virus or hybrids thereof. Even more preferable is a virus comprising H5N1 avian influenza virus.

Preferably, the surface component comprises a coat protein such as but not limited to a surface antigen. Preferably said surface antigen is a hemagglutinin. A multivalent vaccine comprises coat proteins from different strains of virus or different cell surface components from one or more viruses may be used as actives in the preparation of vaccines.

In one embodiment of the present invention, the coat proteins of viruses such as the influenza virus are expressed in vitro. Expression of coat proteins in E. coli, viruses, yeast, or mammalian cells is preferred. Coat proteins such as hemagglutinins are then subjected to treatment with Z. officinale rhizome.

In another embodiment, viruses such as the influenza virus are generated by any convenient way, such as but not limited to recombinant means; preferably by a reverse genetics system, whereby generation of influenza virus is entirely from cloned cDNAs. Extracts of virus comprising coat proteins such as hemagglutinins are then subjected to treatment with Z. officinale rhizome.

Typically, the vaccines of the present invention are prepared as injectibles, either as liquid solutions or suspensions; solid forms suitable for solution in, or suspension in, liquid prior to injection may also be prepared. The preparation may also be emulsified. The active immunogenic ingredients are often mixed with excipients that are pharmaceutically acceptable and compatible with the active ingredient. Such excipients are, for example, water, saline, dextrose, glycerol, ethanol, or the like and combinations thereof. In addition, if desired, the vaccine may also contain minor amounts of auxiliary substances such as wetting or emulsifying agents, pH buffering agents and/or adjuvants that enhance the effectiveness of the vaccine, for example aluminium hydroxide.

The quantity of vaccine to be administered may depend on the subject to be treated, inclusive of age, sex, weight and general health condition thereof. In this regard, precise amounts of the agent(s) for administration will depend on the judgement of the practitioner.

In order that the invention be readily understood and put into practical effect, particular preferred embodiments will now be described by way of the following non-limiting examples.

### Example 1

### Zingibain Inhibits Influenza Viruses

The cell-membrane proteins of viruses, such as neuraminidase and hemagglutinin of the 5 influenza virus, are proline rich with multiple sites for hydrolysis by Zingibain. These proteins are essential for the infection process. Their cleavage inhibits the viral infection, proliferation and removes the antigenicity of the super-antigen, hemagglutinin protein, and the associated T-cell release of damaging high levels of cytokines.

Neuraminidases, including the N1 variant found in H5N1 avian influenza have multiple potential sites for Zingibain hydroloysis adjacent to pro-3, 48, 120, 154, 167, 169, 198, 246, 272, 283, 302, 326, 328, 340, 377, 410, 420, 431, 458 of SEQ ID NO:3 as shown below. These include potential sites on the protein loops adjacent to the amino acids that stabilise the binding of the previous inhibitors and the host cell sialic acid to the enzyme.

| Neuraminidase N1 | |
|---|---|
| 1 | m [symbol] p [symbol] qkitti gsicmvigiv slmlqignii siwvshsiqt gnqhqa [symbol] p [symbol] n qsiityennt |
| 61 | wvnqtyvnis ntnfltekav nlvtlagnss lcpisgwavy skdngirigs kgdvfvir [symbol] p |
| 121 | fiscshlecr tffltqgall ndkhsngtvk dr [symbol] p [symbol] rtlms cpvgeap [symbol] py nsrfesvaws |
| 181 | asachdgtsw ltigisgp [symbol] n gavavlkydg iitdtikswr nnilrtqese cacvngscft |
| 241 | vmtdgp [symbol] ngq asykifriek gkvvksaeln ap [symbol] yhyeecs cyp [symbol] ageitc vcrdnwhgsn |
| 301 | [symbol] pwvsfnqnl eyrigyicsg vfgd [symbol] p [symbol] p [symbol] d gtgscgpv [symbol] p [symbol] gaygikgfs frygngvwig |
| 361 | rtkstnsrsg femiw [symbol] p [symbol] gw tgtdsnfsvk qdivaitdws gysgsfvq [symbol] p [symbol] ltgldci [symbol] p |
| 421 | cfwvelirg [symbol] p [symbol] estiwtsg ssisfcgvns dtvgwswp [symbol] g aelpftidk |

PROLINES ADJACENT TO HYDROPHILIC RESIDUES

[symbol]

[symbol]

[symbol]

[symbol]

[symbol]

[symbol]

Hemagglutinins, including the H5 protein variants found in the Vietnam and Japan/China, and earlier in Singapore H5N1 avian influenza have the following reported structures also set out in SEQ ID NOs:4, 5 and 6, respectively:

| Vietnam H5 1^{st} line; Japan/China H5 2^{nd} line; Singapore H5 3^{rd} line | |
|---|---|
| 1 | MEKIVLLFAI VSLVKSDQIC IGYHANNSTB QVDTIMEKNV TVTHAQDILE KTHNGKLCDL |
| 1 | MEKIVLLLAI VSLVKSDQIC IGYHANNSTE QVDTIMEKNV TVTHTQDILE KKHNGKLCDL |
| 1 | DQIC IGYHANNSTE QVDTIMEKNV TVTHAQDILE KTHNGKLCDL |
| 61 | DGV [symbol] PLILRD CSVAGWLLG [symbol] PMCDEFINVP [symbol] WSYIVEKAN PVNDLCY [symbol] GD FNDYEELKHL |
| 61 | DGV [symbol] PLILRD CSVAGWLLG [symbol] PMCDEFINVP [symbol] WSYIVEKAS PANGLCY [symbol] GD FNDYEELKHL |
| 61 | NGV [symbol] PLILRD CSVAGWLLG [symbol] PMCDEFLNVP [symbol] WSYIVEKDN PVNGLCYPED FNDYEELKHL |
| 121 | LSRINHFEKI QIIPKSSWSS HEASLGVSSA C [symbol] YQGKSSFF RNVVWLIKKN STYP [symbol] IKRSY |
| 121 | LSRINHFEKI QIIPKSSWSN HEASSGVSSA C [symbol] YQGRSSFF RNVVWLIKKN GAYP [symbol] IKRSY |
| 121 | LSSTNHFEKI RIIPRSSWSN HDASSGVSSA C [symbol] YNGRSSFF RNVVWLIKKN NAYP [symbol] IKRSY |
| 181 | NNTNQEDLLV MWGIH [symbol] P [symbol] DA AEQTKLYQ [symbol] P [symbol] TYISVGTSTLNQRLVPRIA TRSKVNGQSG |
| 181 | NNTNQEDLLV LWGIH [symbol] P [symbol] DA AEQTKLYQ [symbol] P [symbol] TYISVGTSTLNQRLVPKIA TRSKVNGQSG |
| 181 | NNTNQEDLLI LWGIH [symbol] P [symbol] DA AEQTKLYQ [symbol] P [symbol] TYVSVGTSTLNQRSVPEIA T [symbol] P [symbol] VNGQSG |
| 241 | RMEFFWTIL [symbol] P [symbol] DAINFESN GNFIAP [symbol] YAY KIVKKGDSTI MKSELEYGNCNTKCQ [symbol] PMGA |
| 241 | RMEFFWTIL [symbol] P [symbol] DAINFESN GNFIAP [symbol] YAY KIVKKGDSAI MKSELEYGNCNTKCQ [symbol] PMGA |
| 241 | RMEFFWTIL [symbol] P [symbol] DAINFESN GNFIAP [symbol] YAY KIVKKGGSAI MKSGLEYGNCNTKCQ [symbol] PMGA |
| 301 | INSSM [symbol] FHNI [symbol] PLTIGE [symbol] P [symbol] YVKSNRLVLA TGLRNSP [symbol] RE RRRKKRGLFGAIAGFIEGGW |
| 301 | INSSM [symbol] FHNI [symbol] PLTIGE [symbol] P [symbol] YVKSNRLVLA TGLRNSP [symbol] RE RRRKKRGLFGAIAGFIEGGW |
| 301 | INSSM [symbol] FHNI [symbol] PLTIGE [symbol] P [symbol] YVKSGRLVLA TGLRNVP [symbol] RE T GLFGAIAGFIEGGW |
| 361 | QGMVDGWYGYHHSNEQGSGYAADKESTQKA IDGVTNKVNS IIDKMNTQFEA VGREFNNLE |
| 361 | QGMVDGWYGYHHSNEQGSGYAADKESTQKA IDGVTNKVNS IIDKMNTQFEA VGREFNNLE |
| 361 | QGMVDGWYGYHHSNEQGSGYAADKESTQKA IDGTTNKVNS IIDKMNTQFEA VGKGFNNLE |
| 421 | RRIENLNKKM EDGFLDVWTY NAELLVLMEN ERTLDFHDSN VKNLYDKVRLQLRDNAKELG |
| 421 | RRIBNLNKKM EDGFLDVWTY NAELLVLMEN ERTLDFHDSN VKNLYDKVRLQLRDNAKELG |
| 421 | RRIENLNKKM EDGFLDVWTY NAELLVLMEN ERTLDFHDSN VKNLYDKVRLQLRDNAKELG |
| 481 | NGCFEFYHKC DNECMESVRN GTYDYP [symbol] YSE EARLKREEIS GVKLESIGIY QILSIYSTVA |
| 481 | NGCFEFYHKC DNECMESVRN GTYDYP [symbol] YSE EARLNREEIS GVKLESIGTY QILSIYSTVA |
| 481 | NGCFEFYHKC DNECMESVKN GTYDYP [symbol] YSB EARLNREEIS GV |
| 541 | SSLALAIMVA GLSLWMCSNG SLQCRICI |
| 541 | SSLALAIMVA GLSLWMCSNG SLQCRICI |

NON-CONSERVED RESIDUES
NON-CONSERVED HYDROPHILIC RESIDUES ADJACENT TO PROLINE

[symbol]

[symbol]

[symbol]

[symbol]

[symbol]

[symbol]

PROLINES ADJACENT TO HYDROPHILIC RESIDUES

[symbol]

[symbol]

[symbol]

[symbol]

[symbol]

[symbol]

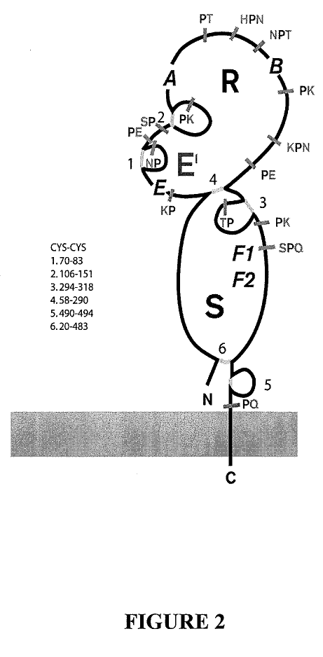

For these three variants of H5 hemagglutinin, the prolines are conserved except for the additional pro-233, with the hydrophilic groups arginine and lysine adjacent to it, in the Singapore variant replacing serine in the other two, with the adjacent hydrophilic amino acids conserved, except for pro-101 with asn-100 for the Vietnam and Singapore variants and ser-100 for the Japan/China protein, pro-108 with the non-hydrophilic glycine for the Vietnam and Japan/China variants and the hydrophilic glutamate for the Singapore variant giving an additional site for hydrolysis, pro-134 with lysine for the Vietnam and Japan/China variants and with arginine for the Singapore protein, and for pro-337 which has glutamine following it for all three but with serine before the proline for Vietnam and Japan/China and valine for the Singapore variant. The target prolines for potential Zingibain hydrolysis are: pro-65, 81, 90, 101, 108 (Singapore only), 134, 174, 197, 210, 227, 233 (Singapore only) 251, 266, 297, 312, 319, 337, 506 as shown in FIG. 2 for the Japan/China variant.

Although the various H5 structures show significant mutations, the potential sites for Zingibain hydrolysis are largely conserved and number at least 15 sites that Zingibain has an excellent opportunity to hydrolyse the H5 protein.

The H3N2 influenza a virus subtype has a similar number of potential sites for Zingibain hydrolysis even though the protein structure does not have a high level of homology with the H5 structure. The following sequences compare the H5 structure on the 1^{st} line with a H3 structure (SEQ ID NO:7) on the 2^{nd} line:

| | |
|---|---|
| 1 | XXLXXXDQIC IGYHANNSTE QVDTIMEKNV TVTHXQDILE KXHNGKLCDL-- |
| | QDL [symbol] GNDNST ATLCLGHHAV P [symbol] GTLVKT IT DDQIEVTNAT ELVQSSSGKICN |
| 61 | XGV [symbol] PLILRD CSVAGWLLG [symbol] PMGDEFXNVP [symbol] WSYIVEKXZ PXNXLCYPZ D FNDYEELKHL |
| | NP [symbol] RI LDGID CTL I DA LLG [symbol] P [symbol] CDVFQNE- TWDLFVERSK-AFSN CY [symbol] YD VP [symbol] YASL--- |
| 121 | LSXXNHFEKI XIIPZSSWSN HXASXGVSSA C [symbol] YXGXSSFF RNVVWLIKKN XXYP [symbol] IKRSY |
| | RSLVASSGTL EFITEGFTWT GVIQNGGSNA CKRG [symbol] GSGFF SRLNWLTKSGSTY [symbol] VLNVTM |
| 181 | NNTNQEDLLX XWGIH [symbol] P [symbol] DA AEQTKLYQNP [symbol] TYXSVGTSTLNQRXVPZIAT [symbol] Z [symbol] VNGQSG |
| | P [symbol] NDNFDKLY IWGIH [symbol] P [symbol] TN QEQTSLYVQASGRVTVSTRRSQQTII P [symbol] IGS [symbol] PWVRGLSS |
| 241 | RMEFFWTIL [symbol] P [symbol] DAINFESN GNFIAP [symbol] YAY KIVKKGXSXI MKSXLEYGNCNTKCQ [symbol] PMGA |
| | SRISIYWTIV [symbol] PGDVLVINSN GNLIAPRGTF KMRT-GKSSI MRS DA [symbol] IDT C I SE C I [symbol] PNGS |
| 301 | INSSM [symbol] FHNI [symbol] PLTIGE [symbol] P [symbol] YVKSXRLVLA TGLRNXP [symbol] RE X GLFGAIAGFIEGGW |
| | IP [symbol] D [symbol] PFQNV NKITYGA [symbol] P [symbol] TVKQNTLKLATGMRNVPEKQT GLFGAIAGFIENGW |
| 361 | QGMVDGWYGYHHSNEQGSGYAADKESTQKA IDGXTNKVNSIIDKMNTQFEAVGXXFNNLE |
| | EGM I DGWYGFRHQNSEGTGQAADLKSTQAA IDQINGKL NRVIEKTNEKFHQI EKEFSEVE |
| 421 | RRIENLNKKM EDGFLDVWTY NAELLVLMEN ERTLDFHDSN VKNLYDKVRLQLRDNAKELG |
| | GRIQDLEKYV EDTKIDLWSY NAELLVALEN QHTIDLTDSE MNKLFEKTRRQLRENAEEMG |
| 481 | NGCFEFYHKC DNECMESVKN GTYDYP [symbol] YSE EARLXREEISGV |
| | NGCFKIYHKC DNACIESIRN GTYDHDVYRD EALNNRFQIKG |

[symbol]

[symbol]

[symbol]

[symbol]

[symbol]

[symbol]

PROLINES ADJACENT TO HYDROPHILIC RESIDUES

[symbol]

[symbol]

[symbol]

[symbol]

[symbol]

[symbol]

X IS A NON-CONSERVED RESIDUE IN THE THREE H5 PROTEINS
Z IS A NON-CONSERVED RESIDUE IN A ZINGIBAIN HYDROLYSIS SITE

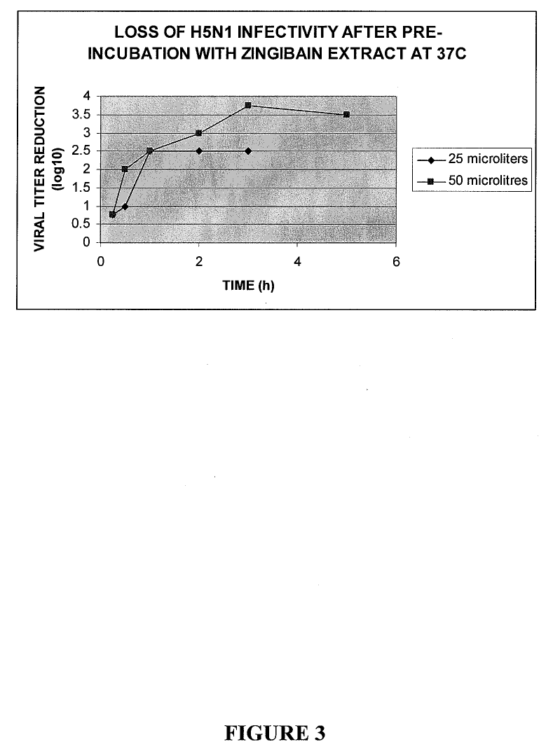

An antiviral drug assay with Madin-Darby Canine Kidney (MDCK) cells for testing the inhibitory activity of a drug against viruses was used for a Zingibain extract (1400 units activity/mL) with the H3N2 subtype influenza-A and H5N1 (Vietnam) avian influenza. Different amounts of the extract were mixed with 10 μL of 500,000 H3N2 or 10 μL of 100,000 H5N1 infectious units for different times at 37° C. Medium was then added to dilute the ginger-virus mixture to 1 mL. Residual virus viability was tested on 96-well-plates with confluent MDCK cells. Inhibition effects were increased from 1log10 to 1.5log10 and to 2.5log10 when the volume of Zingibain extract was increased from 10 microlitres to 25 microlitres to 50 microlitres for H3N2 and from 1log10 to 1.5log10 and to 2log10 for the same volumes for the H5N1 virus for 30 minute digestions. The loss of infectivity with incubation time at 37° C. for H5N1 is given in FIG. 3 for 25 microlitres and 50 microlitres of extract.

### Example 2

### Zingibain Inhibits Ross River Virus

An antiviral drug assay for testing the inhibitory activity of a drug against viruses was also used for Zingibain with the mosquito-borne virus, Ross River Virus (RRV), at a dilution of 10⁻⁵ and 10^{-6.} The virus was mixed with Zingibain at 7.5 Units activity/mL, and allowed to incubate at pH 7.2 for 2 hours. This was added to a confluent monolayer of Vero cells. The plaques produced by the virus were counted.

TABLE 2
Plaque assay of Ross River Virus incubated
with Zingibain (7.5 Units/mL) In Vero cells

| | Av. No. Plaques | Av. No. Plaques |
|---|---|---|
| RRV | with Zingibain | without Zingibain |
| −5 | 46 | 140 |
| −6 | 4 | 20 |

Higher concentrations of Zingibain could not be used in this type of assay, which relies on the cells being adhered to a glass surface, because Zingibain rounds up cells such as Vero cells from a surface. At 7.5 Units activity/mL, Zingibain inhibited RRV by up to 80%.

### Example 3

### Zingibain Inhibits Murray Valley Encephalitis

The ability of Zingibain at 330 Units/mL in serum-free media to inhibit Murray Valley Encephalitis replication was studied in Vero cells previously infected for 1 hour with a range of dilutions of the virus at 37° C. The dilution of the virus at which the virus was detected in 50% of the wells was 10⁻⁶ with no Zingibain and 10^{-5.3} for 330 Units/mL Zingibain. This equates to approximately a 5-fold inhibition of virus replication.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Choi et al, Biochem. 38:11624-11633, 1999

1. A method of treating influenza virus infection comprising administering an extract of a molecular component of a rhizome from a species of Zingiber or fraction thereof to a subject in need thereof wherein the extract or fraction thereof comprises a cysteine protease which hydrolyzes proline-containing influenza virus proteins.

2. The method of Claim 1, wherein the species of Zingiber is selected from the group consisting of Zingiber officinale, Zingiber mioga, Zingiber cassumunar and Zingiber zerumbet.

3. The method of Claim 2, wherein the species of Zingiber is Zingiber officiniale.

4. The method of claim 1, wherein the cysteine protease hydrolyzes peptide bonds in an influenza virus protein between an amino acid following a proline and the next amino acid thereafter in amino acid sequence from the N-terminal end.

5. The method of Claim 4, wherein the amino acid residue before or after proline residue is a hydrophilic amino acid residue selected from the group consisting of glutamine, arginine, lysine, asparagine, glutamic acid and aspartic acid.

6. The method of Claim 1 wherein the influenza virus is avian influenza virus A.

7. A method of treating an infection caused by an influenza virus comprising administering an extract of Zingiber comprising a cysteine protease which hydrolyzes proline-containing influenza virus proteins to a subject in need thereof.

8. The method of Claim 7 wherein the infection is caused by avian influenza virus.

9. A method of treatment of a subject infected with an influenza virus administering to the subject a therapeutically effective amount of an extract of a species of Zingiber or a molecular component thereof which extract or molecular component comprises a cysteine protease which hydrolyzes proline-containing influenza proteins.

10. The method of Claim 9 wherein the influenza virus is avian influenza virus.
